(19) United States
(12) Patent Application Publication
(10) Pub. No.: US 2009/0004051 A1
(43) Pub. Date: Jan. 1, 2009

(54) Processing of Documents with Medical and Other Waste

(75) Inventors: Russell A. Firestone, Washington, DC (US); James R. Harkess, Studio City, CA (US); William S. Langhorne, Falls Church, VA (US); Nord S. Sorensen, Amarillo, TX (US)

Correspondence Address: MINTZ, LEVIN, COHN, FERRIS, GLOVSKY AND POPEO, P.C;ATTN: PATENT INTAKE CUSTOMER NO. 64046, ONE FINANCIAL CENTER, BOSTON, MA, 02111, US

(73) Assignee: Sanitec Industries, Inc., Washington, DC (US)

(21) Appl. No.: 11/578,070

(22) PCT Filed: Mar. 11, 2005
(86) PCT No.: PCT/US2005/008100

§ 371 Date: May 6, 2008

Related U.S. Application Data
(60) Provisional application No. 60/560,972, filed on Apr. 12, 2004.

(51) Int. Cl. A61L 2/20; A61L 2/04; A61L 2/08; A61L 2/10; B02C 18/00

(52) U.S. Cl. 422/28; 422/1; 422/184.1; 241/27

(57) ABSTRACT

The invention relates to methods and apparatus for processing documents with medical and other waste. The method includes the steps of supplying the documents to an apparatus (1, 6) adapted to shred documents and medical waste, shredding the documents, and discharging the shredded documents from the apparatus (1). The apparatus (1) has little or no liquid effluent and little or no toxic air emissions. Also included is a system and method for tracking medical or toxic waste including a first container (13) adapted to hold untreated medical waste, the container having a wireless tracking device (170) attached thereto. The system also includes a tracking station capable of monitoring the movement of the medical waste by tracking the wireless tracking device (170).

### FIELD OF THE INVENTION

The present invention is directed generally to the destruction of documents and specifically to the destruction of documents in combination with medial and other waste.

The present invention is directed generally to the tracking of waste and specifically to the tracking of medical and other toxic or hazardous waste.

### BACKGROUND OF THE INVENTION

Due to concern over spread of individually identifiable health information and the perceived need for the protection of the privacy this health information, Congress passed the Health Insurance Portability and Accountability Act (HIPAA) in 1996. After several rounds of notice and comment, the U.S. Department of Health and Human Services (HHS) passed privacy rules to implement this Act in 2002. The goal of these rules is to assure that an individual's health information is properly protected while allowing the flow of health information needed to provide and promote high quality health care.

The HHS rules significantly limit the disclosure of an individual's health information—called “protected health information” in the rules—by organizations subject to the rules. Under the rules, acceptable safeguards for protecting the privacy of protected health information include shredding any documents having protected health information or keeping the records under lock and key. Additionally, some health care organizations destroy their documents by incinerating them.

In addition to the generation of a large number of documents having protected health information, health care organizations such as hospitals and clinics generate large amounts of medical waste. Typically, these organizations dispose of these two types of waste in completely distinct ways. That is, the organizations typically engage one waste disposal organization to treat the medical waste and another organization to shred the medical documents. This is both inefficient and expensive.

While it is possible to incinerate both documents and medical waste together, this typically results in the generation of a large amount of toxic emissions. In fact, while incinerators may reduce the volume of solid waste, they typically create toxic emissions, especially when incinerating plastics. In fact, incinerators are the largest source of dioxins, one of the most toxic chemicals known to science. Thus, it would be advantageous to have a single method capable of destroying both documents and medical waste that was efficient, inexpensive and produced little or no toxic air emissions.

The generation and destruction of industrial and medical waste is a serious problem that has become increasingly more important each year as the volume of waste generated continues to increase. This is especially true for medical waste as it typically includes toxic and/or infectious matter.

Although there are many regulations that govern the actual destruction of industrial and medical waste, historically there was very little regulation governing the tracking of this waste. This lack of regulation has occasionally resulted in abuse as the waste was improperly and/or illegally dumped rather than destroyed. In fact, because of widespread mismanagement, medical wastes began washing ashore along the Atlantic Coast during the summer of 1988.

In response to the medical waste disaster of 1988, the United States Congress passed the Medical Waste Tracking Act. The Act requires the use of a tracking form for all facilities that generate over 50 pounds a month of medical waste and for all shipments over 50 pounds. On the form, the generator must put the facility name and address, the identity of the transporter, the intended destination facility and the waste category. Additionally, the Act requires that medical waste producers segregate waste at the point of generation, place waste in containers that protect waste handlers and the public from exposure, and label the waste containers appropriately.

The cost of compliance with this method of tracking is relatively high and has resulted in widespread noncompliance. Because of the extent of noncompliance and the paucity of funds for enforcement, the EPA has typically ignored the problem. This has only encouraged others to ignore the statute, further increasing the problem. Thus, it would be advantageous to have a cheaper method of tracking medical and industrial waste that is easier to monitor and enforce.

### SUMMARY OF THE INVENTION

The present invention provides a method of destroying documents comprising supplying the documents to an apparatus adapted to shred documents and medical waste, the apparatus having little or no liquid effluent and little or no toxic air emissions, shredding the documents, and discharging the shredded documents from the apparatus.

The present invention provides a method of tracking medical waste comprising monitoring the movement of a first container having a wireless tracking device attached thereto from a waste generating facility to a waste treatment facility using the wireless tracking device.

The present invention also includes a system for tracking medical waste comprising a first container adapted to hold untreated medical waste, the container having a wireless tracking device attached thereto, and a tracking station capable of monitoring the movement of the medical waste by tracking the wireless tracking device.

### BRIEF DESCRIPTION OF THE DRAWINGS

The foregoing and other features, aspects and advantages of the present invention will become apparent from the following description, appended claims and the exemplary embodiments shown in the drawings, which are briefly described below. It should be noted that unless otherwise specified like elements have the same reference numbers.

FIG. 1 is a schematic illustration of an apparatus suitable for use in methods according to an embodiment of the invention.

FIG. 2 is a schematic illustration of a second apparatus according to another embodiment of the invention.

FIG. 3 is a schematic illustration of third apparatus according to another embodiment of the invention.

FIG. 4 is a schematic illustration of a fourth apparatus according to another embodiment of the invention.

FIG. 5 is a schematic illustration of a wireless tracking system according to one embodiment of the invention.

FIG. 6 is a schematic illustration of a container having a wireless tracking device used in a method according to an embodiment of the invention.

FIG. 7 is a schematic illustration of another embodiment of the invention.

### DETAILED DESCRIPTION OF THE PREFERRED EMBODIMENTS

The present inventors have discovered that it is possible to dispose of both medical waste and documents such as those subject to HIPAA rules in a single process while produced little or no toxic air emissions. In particular, the inventors have discovered, that this may be accomplished by judiciously mixing documents having protected health information with medical waste and supplying the mixture to a microwave based treatment apparatus. When fed at an appropriate rate, the documents may be shredded to a level acceptable under the HHS rules. Further, when held in the microwave apparatus for a sufficient amount of time, the biological waste can be destroyed without the generation of dioxins and other toxic byproducts. Additionally, it is noted that the documents need not be restricted to those containing protected health information. Any and all documents can destroyed in the processes according to the various embodiments of the invention discussed in more detail below.

For purposes of the present disclosure, protected health information includes demographic data that identifies an individual or for which there is a reasonable basis to believe can be used to identify the individual. Typically, demographic data includes (1) the individual's physical health, mental health, and/or medical condition, (2) health care services requested by, sought by, recommended to, administered to, and/or prescribed to the individual, and (3) the billing records for the provision of health care to the individual.

More specifically, demographic data includes: (A) names, (B) geographic subdivisions smaller than a state, where geographic subdivisions smaller than a state includes street address, city, county, precinct, zip code, and their equivalent geocodes, (C) elements of dates for dates directly related to the individual, where elements of dates includes birth date, admission date, discharge date, date of death and age, (D) telephone numbers, (E) fax numbers, (F) electronic mail addresses, (G) social security numbers, (H) medical record numbers, (I) health plan beneficiary numbers, (J) account numbers; (K) certificate/license numbers, (L) vehicle identifiers and serial numbers, (M) license plate numbers, (N) device identifiers and serial numbers, (O) web universal resource locators, (P) internet protocol address numbers, (Q) biometric identifiers, where biometric identifiers includes finger and voice prints, and (R) full face photographic images.

For the purposes of this disclosure, medical waste may include, but is not limited to:

(1) cultures and stocks of infectious agents and associated Biologicals, including cultures from medical and pathological laboratories, cultures and stocks of infectious agents from research and industrial laboratories, wastes from the production of biologicals, discarded live and attenuated vaccines, and culture dishes and devices used to transfer, inoculate, and mix cultures;
(2) pathological wastes, including tissues, organs, and body parts that are removed during surgery or autopsy;
(3) waste human blood and products of blood, including serum, plasma, and other blood components;
(4) sharps that have been used in patient care or in medical, research, or industrial laboratories, including hypodermic needles, syringes, pasteur pipettes, broken glass, and scalpel blades;
(5) contaminated animal carcasses, body parts, and bedding of animals that were exposed to infectious agents during research, production of biologicals, or testing of pharmaceuticals;
(6) wastes from surgery or autopsy that were in contact with infectious agents, including soiled dressings, sponges, drapes, lavage tubes, drainage sets, underpads, and surgical gloves;
(7) laboratory wastes from medical, pathological, pharmaceutical, or other research, commercial, or industrial laboratories that were in contact with infectious agents, including slides and cover slips, disposable gloves, laboratory coats, and aprons;
(8) dialysis wastes that were in contact with the blood of patients undergoing hemodialysis, including contaminated disposable equipment and supplies such as tubing, fitters, disposable sheets, towels, gloves, aprons, and laboratory coats;
(9) discarded medical equipment and parts that were in contact with infectious agents;
(10) biological waste and discarded materials contaminated with blood, excretion, excudates or secretion from human beings or animals who are isolated to protect others from communicable diseases;
(11) radioactive waste used industrially and in medical procedures as well as chemical waste used in the production of reagents used in laboratories and medical facilities; and
(12) such other waste material that results from the administration of medical care to a patient by a health care provider and is found by the administrator of the EPA to pose a threat to human health or the environment.

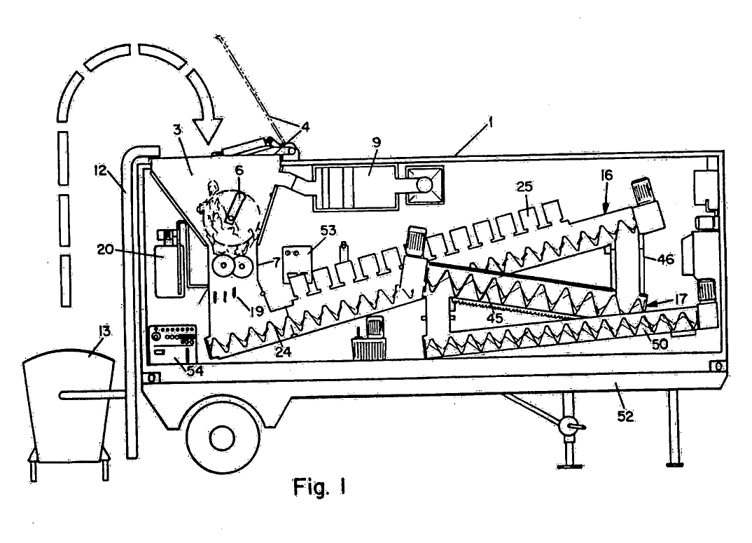

FIG. 1 illustrates a waste treating apparatus 1 suitable for use in a method of destroying documents and treating medical waste according to a first embodiment of the invention. The waste treating apparatus 1 used in this embodiment is similar to one disclosed in U.S. Pat. No. 5,270,000, which is hereby incorporated by reference. Waste contained in a waste container 13 is delivered to a lift-and-tip mechanism 12. The lift-and-tip mechanism 12 lifts the waste container 13 to the opening of the loading chamber 3 and dumps the waste therein. After dumping the waste into the loading chamber 3, a cover 4 having fluid-tight seal is closed to prevent the escape of germs into the atmosphere. To further inhibit the loss of germs into the atmosphere, the waste treating apparatus 1 includes a suction system 9. The suction system 9 includes a suction pump and at least one filter to capture airborne germs.

In this embodiment of the invention, the documents and medical waste are mixed in the waste container 13 prior to lifting and tipping into the loading chamber 3. However, it is not necessary to premix the documents and medical waste. In other embodiments of the invention, waste containers 13 having only medical waste are alternately loaded with waste containers 13 having only documents. In this manner, the documents and medical waste mix in the waste treating apparatus 1. Further, in these embodiments, it is not necessary to alternately load one waste container 13 of medical waste for each waste container 13 of documents. That is, the ratio of documents to medical waste may be adjusted to ensure proper mixing and destruction of both the documents and the medical waste.

Documents and medical waste dumped into the waste treating apparatus 1 descend in the loading chamber 3 to a blade 6. The blade 6 performs two functions. First, it aids in drawing the waste down the loading chamber 3. Additionally, it performs a first step of cutting the waste into smaller pieces.

Below the blade 6 is a waste comminutor 7. Waste cut by the blade 6 falls to the waste comminutor 7. The waste comminutor 7 shreds the chopped waste to fine pieces, pieces small enough to satisfy the HHS requirements for shredding documents having protected health information. Typically, the waste comminutor 7 includes a pair of counter-rotating blades, however, any suitable shredding mechanism may be used.

After being shredded by the waste comminutor 7, the finely shredded mixture of documents and medical waste falls from the waste comminutor 7 past a series of spray nozzles 19, which spray the shredded waste mixture with water. The water may be supplied from a water tank 20 or from a water line connected to the waste treating apparatus 1. The amount of water sprayed depends on the volume of waste mixture dropping past the spray nozzles 19 and the desired amount of moistening. Preferably, the waste mixture is uniformly moistened.

The moistened waste mixture is then loaded on a conveying helix 24 that conveys the moistened waste mixture through a microwave chamber 16 having a series microwave sources 25. The microwave sources 25 heat the moistened waste mixture to a temperature hot enough to kill bacteria and viruses. In addition to conveying the moistened waste mixture, the conveying helix 24 further mixes the documents and the medical waste to create a more homogeneous mixture of waste.

To ensure that all of the bacteria and viruses are dead, the heated moistened waste mixture is passed from the microwave chamber 16 through a heated passage 46 to a heat maintenance chamber 17. The moistened waste mixture is slowly advanced through the heat maintenance chamber 17 by use of a conveying helix 45. The rate of conveyance through the heat maintenance chamber 17 is adjusted so that the waste remains at a sufficiently elevated temperature for a sufficient amount of time to kill all of the viruses and bacteria. As with the conveying helix 24, the conveying helix 45 further mixes the documents and the medical waste to create a more homogeneous mixture.

After spending sufficient time in the heat maintenance chamber 17 to kill all of the viruses and bacteria, the moistened waste mixture is passed to an unloading mechanism 50. The unloading mechanism 50 further mixes the moistened waste mixture and advances it through the waste treating apparatus 1 to a treated waste container (not shown). Additionally, as there is no further heat added to the waste, the hot waste mixture undergoes partial cooling while being advance by unloading mechanism 50.

In the embodiment of the invention illustrated in FIG. 1, the waste treating apparatus 1 is located on a motor vehicle trailer 52. Thus, the waste treating apparatus 1 of this embodiment is portable. In other embodiments of the invention, the waste treating apparatus 1 is erected in a permanent fashion, preferably adjacent to or nearby the waste generating facility. However, a permanent waste treating apparatus 1 may erected at a location remote to the waste generating facility.

To control process, the waste treating apparatus 1 may include a process-control computer 54. Additionally, a space heating system 53 may be included to provide additional heat to the waste treating apparatus 1.

The waste treating apparatus 1 according to the above embodiments of the invention require very little water for operation. Further, the temperature of operation is low enough so that harmful dioxins are not generated. Thus, there is no large runoff of water typically associated with an autoclave, nor large toxic emissions typically associate with incinerators. In fact, the waste treating apparatus 1 used in the above described methods have little or no liquid effluent and little or no toxic air emissions. Further, the processed waste discharged from the waste treating apparatus 1 is suitable to go directly to a landfill or to a recycling center without additional treatment.

In addition to traditional medical waste, many other infectious or toxic materials can be treated in conjunction with documents by the methods of the present invention. Other embodiments of the invention include diseased animal carcasses, diseased human bodies and natural disaster victims. Additionally, documents can be mixed with contaminated food in the waste treating apparatuses 1. In still another embodiment, the documents may be mixed with illegal drugs or expired pharmaceuticals.

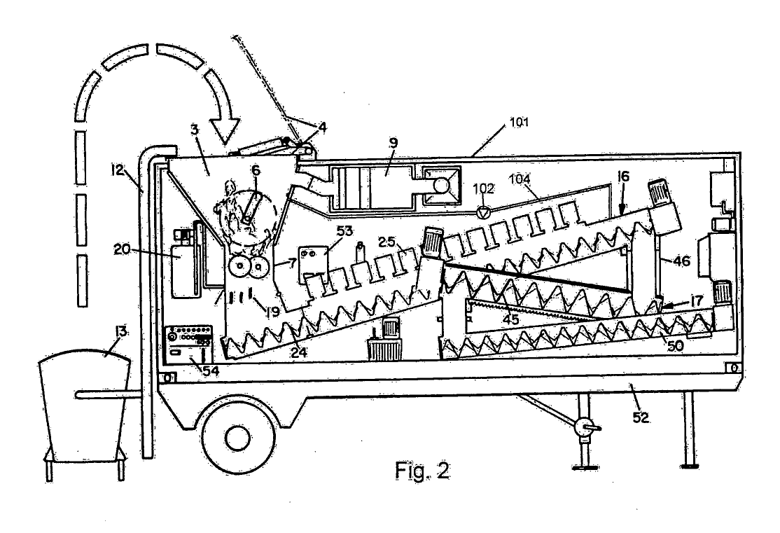

FIG. 2 illustrates a waste treating apparatus 101 according to another embodiment of the invention. The waste treating apparatus 101 according to this embodiment of the invention is similar to the waste treating apparatus 1 of the previous embodiments, however it includes the ability to preheat waste in the loading chamber 3. That is, the waste treating apparatus 1 includes an air recycle pipe 104 that extends from the exit of the microwave chamber 116 to the loading chamber 3. Also included is a pump 102 which pumps hot air from the exit of the microwave chamber 116 to the loading chamber 3. In this manner, hot air from the exit of the microwave chamber 16 may be used to heat the waste as it enters the loading chamber 3. As may be appreciated by one of ordinary skill in the art, the preheat mechanism of this embodiment may be used in combination with any of the other disclosed embodiments even though it is only illustrated in this embodiment.

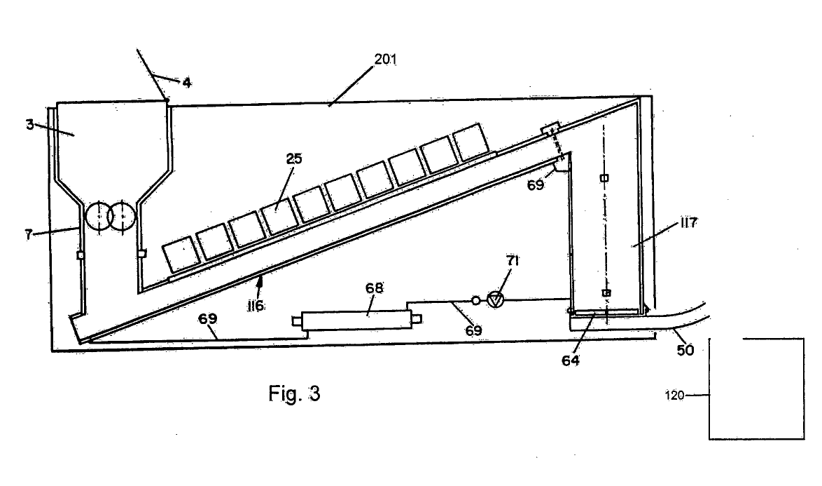

FIG. 3 illustrates another embodiment of the invention. In this embodiment of the invention, the waste treating apparatus 201 is configured for continuous treatment of waste. That is, the medical waste and documents are continuously feed into the waste treating apparatus 201. This may be accomplished through a conveyor system (not shown) or any other continuous feed mechanism known in the art.

Initially, the waste is prevented from exiting the waste treating apparatus 201 by a cover 64 which blocks the path from the temperature maintenance chamber 117 to the unloading mechanism 50. When the waste has spent a sufficient amount of time in the temperature maintenance chamber 117 to kill all of the bacteria and viruses, the cover 64 is removed, allowing the waste to pass from the temperature maintenance chamber 117 to the unloading mechanism 50.

An additional aspect of this embodiment concerns the unloading mechanism 50. In this embodiment, the unloading mechanism 50 has a diameter that is less than the diameter of the microwave chamber 116. This results in a partial compaction of the waste while exiting the waste treating apparatus 201. To further increase compaction, a compactor 120 may optionally be added to the exit of the waste treating apparatus 201.

Still another aspect of this embodiment is the inclusion of a second, conductive, heating mechanism. This aspect includes a fluid reservoir 68, a fluid pump 71 and piping 69. Further, in this embodiment, the microwave chamber 116 and temperature maintenance chamber 117 are double wall chambers having a gap between the walls. Fluid from the fluid reservoir 68 is pumped through the double wall of the temperature maintenance chamber 117, thereby being heated. It then flows through the double walls of the microwave chamber 116. In this manner additional heat is added to the waste as it travels through the microwave chamber 116. As may be appreciated by one of ordinary skill in the art, the conductive heating mechanism of this embodiment may be used in combination with any of the other disclosed embodiments even though it is only illustrated in this embodiment.

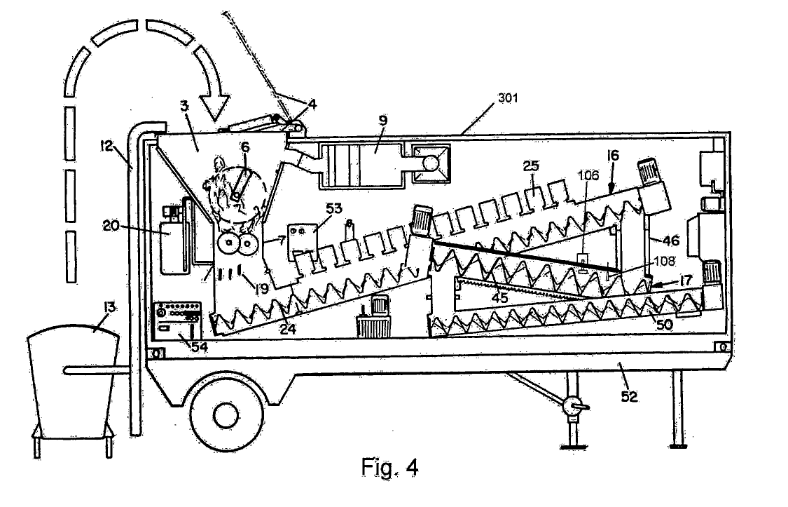

FIG. 4 illustrates additional embodiments of the invention. In this embodiment, the waste treating apparatus 301 includes a secondary radiation source 106 added to the temperature maintenance chamber 17. The secondary radiation source 106 preferably is either a microwave or an infrared heat source. However, X-ray and UV sources may be used as well. In addition to the secondary radiation source 106, the waste treating apparatus 301 may also include an ozone generating electrode 108 either singly or in combination with the secondary radiation source 106. The ozone generated from the ozone generating electrode 108 is highly oxidizing and is known to kill germs. Thus, the addition of the ozone generating electrode 108 increases the likelihood that all of the bacteria and viruses in the medical waste are killed.

The present inventors have determined that by using a wireless tracking system, a method of tracking medical and industrial waste can be implemented that is both cost effective and easy to enforce. In short, by supplying waste generators with containers having a wireless tracking device attached, it is possible to dynamically track the movement of untreated waste to a treatment facility. In one preferred embodiment of the invention, individuals with handheld readers scan the tracking device at various points along the disposal route. The information can then be upload from the handheld reader to a central monitoring station. In another preferred embodiment, the tracking is accomplished by use of GPS technology. When the waste is destroyed, the operator of the waste treatment facility can send a signal to the monitor to indicate the destruction. Optionally, an invoice may be generated and the customer billed. Additionally, the treated waste may be placed in a second container having a wireless tracking device attached and the movement of the treated waste tracked until its final disposition.

For the purposes of this disclosure, medical waste may include, but is not limited to:

(1) cultures and stocks of infectious agents and associated Biologicals, including cultures from medical and pathological laboratories, cultures and stocks of infectious agents from research and industrial laboratories, wastes from the production of biologicals, discarded live and attenuated vaccines, and culture dishes and devices used to transfer, inoculate, and mix cultures;
(2) pathological wastes, including tissues, organs, and body parts that are removed during surgery or autopsy;
(3) waste human blood and products of blood, including serum, plasma, and other blood components;
(4) sharps that have been used in patient care or in medical, research, or industrial laboratories, including hypodermic needles, syringes, pasteur pipettes, broken glass, and scalpel blades;
(5) contaminated animal carcasses, body parts, and bedding of animals that were exposed to infectious agents during research, production of biologicals, or testing of pharmaceuticals;
(6) wastes from surgery or autopsy that were in contact with infectious agents, including soiled dressings, sponges, drapes, lavage tubes, drainage sets, underpads, and surgical gloves;
(7) laboratory wastes from medical, pathological, pharmaceutical, or other research, commercial, or industrial laboratories that were in contact with infectious agents, including slides and cover slips, disposable gloves, laboratory coats, and aprons;
(8) dialysis wastes that were in contact with the blood of patients undergoing hemodialysis, including contaminated disposable equipment and supplies such as tubing, fitters, disposable sheets, towels, gloves, aprons, and laboratory coats;
(9) discarded medical equipment and parts that were in contact with infectious agents;
(10) biological waste and discarded materials contaminated with blood, excretion, excudates or secretion from human beings or animals who are isolated to protect others from communicable diseases;
(11) radioactive waste used industrially and in medical procedures as well as chemical waste used in the production of reagents used in laboratories and medical facilities; and
(12) such other waste material that results from the administration of medical care to a patient by a health care provider and is found by the administrator to pose a threat to human health or the environment.

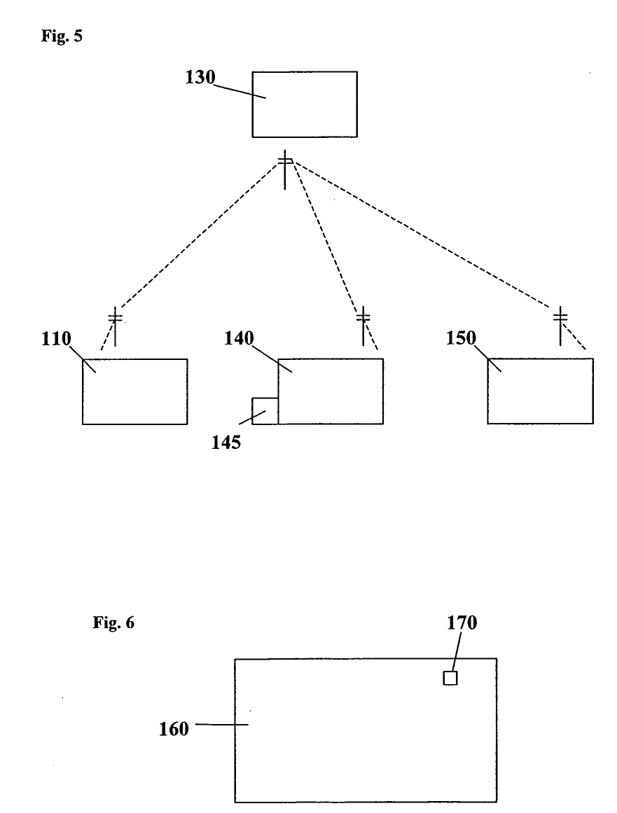

One embodiment of a wireless waste tracking system 100 according to the present invention is illustrated in FIGS. 5 and 6. This embodiment of the invention includes a waste disposal container 160 (FIG. 6) having a wireless tracking device 170. The tracking device 170 may be affixed to the container 160 by relatively permanent method such as welding or gluing. However, the tracking device 170 may be replaceably affixed to the container 160, for example by a snap fit. Indeed, the tracking device can also be simply added to the waste as a component the will end up deposited with the waste at its final destination, for example at a landfill.

The wireless tracking system 100 also includes a tracking station 130. The tracking station 130 is equipped with a computer system (not shown) that stores tracking information as the waste disposal containers 160 are shipped from a waste generator 110 to a waste treatment facility 140. In this embodiment, an operator reads tracking information from the tracking device using a handheld reader (not shown) and downloads the information to the computer at the monitoring station 130. The downloading may be done over the phone lines or through a wireless communications network. Additionally, the movement of the waste disposal containers 160 may also be monitored as they are shipped from the waste treatment facility 140 to a final destination 150 for the waste. Readings with a handheld reader may be taken at each step in this process and the computer log updated throughout the delivery from the initial pickup at the waste generator 110 to the final destination 150.

Typically, the waste generator 110 is a hospital, urgent care center, or medical practice. However, the waste generator 110 may also include a ranch with diseased animals, a farm or orchard with diseased fruit, a customs inspection center with banned products, a factory with toxic waste or any other producer of waste that should be tracked to ensure proper disposal. The waste treatment facility 140 may be a chemical treatment plant, an industrial incinerator or any other facility suitable for destroying or denaturing medical or toxic waste. Typically, the final destination 150 is a municipal dump, landfill or a recycling plant. However, the final destination 150 may be any facility or ground suitable for processing or holding treated waste.

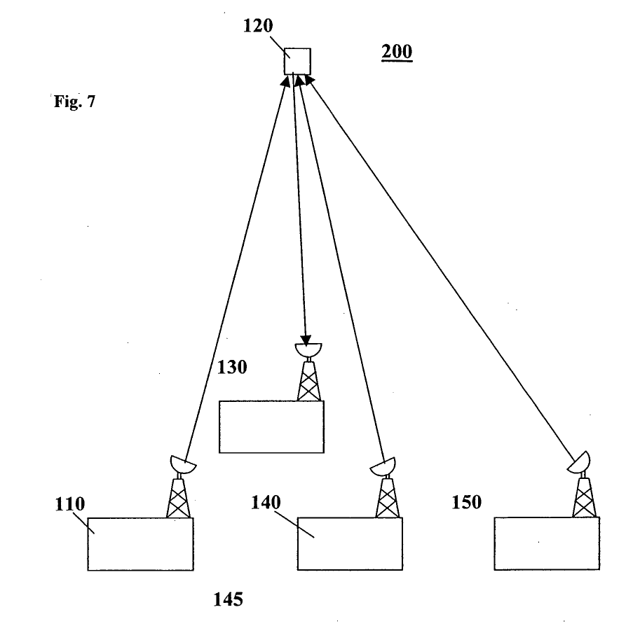

FIG. 7 illustrates another embodiment of the invention. The wireless tracking system 200 also includes a tracking station 130. The tracking station 130 is equipped to continuously monitor the movement of waste disposal containers 160 as they are shipped from a waste generator 110 to a waste treatment facility 140. Further, the tracking station 130 is equipped to continuously monitor the movement of waste disposal containers 160 as they are shipped from the waste treatment facility 140 to a final destination 150 for the waste.

In this embodiment, the wireless waste tracking system 200 typically includes a communications forwarding device 120. The communications forwarding device 120 may be any type of device that can monitor and forward signals from a wireless tracking device 170. The communications forwarding device 120 may be, for example, a satellite or cellular receiving station. In a preferred embodiment of the invention, the tracking device 170 and the communications forwarding device 120 operate in radio frequency and may be integrated into the GPS system. However, the tracking device 170 and the communications forwarding device 120 may operate at any portion of the electromagnetic spectrum, for example microwave frequencies.

Methods according to the present invention will now be discussed. In a first embodiment of the method, the waste generator 110 puts untreated waste (not shown) in a waste disposal container 160 (FIG. 6) having a tracking device 170. When the waste disposal container 160 is full, or the decision is made to dispose of the waste, the waste generator 110 arranges to ship the waste to a waste treatment facility 140. If the first system 100 is used, either an operator at the waste generator 110 or an operator associated with a waste transporter can take an initial reading with a handheld reader and send the data to the tracking station 130.

In a preferred embodiment of the invention, the waste treatment facility 140 includes a weighing apparatus 145 which can determine the weight of the waste without removing it from the waste disposal container 160. In another preferred embodiment, the waste treatment facility 140 also includes a computer (not shown) with billing software. When the waste is destroyed, the computer calculates how much the customer owes based on the weight and type of waste destroyed. The type of waste destroyed may either be read from the tracking device or inputted by an operator at the waste treatment facility 140. Preferably, the computer also generates an electronic invoice/bill confirming the destruction of the waste, the amount of the waste destroyed and the amount owed by the customer.

In still another embodiment of the invention, the treated waste is placed in a second waste disposal container 160 having a tracking device 170 and shipped to a final destination 150. Typically, the final destination is a municipal dump. However, depending on the type of waste, the final destination 150 may be a recycling center. Preferably, the final destination 150 includes a device (not shown) for generating an invoice that confirms receipt of the treated waste. The invoice may also include information indicating if further payment is required or a credit is due the customer.

Additionally, the various medical waste and document destruction embodiments may be combined with the various tracking embodiments. In this manner, the destruction of medical waste and documents and their disposal may be closely and efficiently monitored. Further, the costs may be quickly and efficiently determined and invoiced to the customer as the documents and medical waste are being destroyed and tracked.

The foregoing description of the invention has been presented for purposes of illustration and description. It is not intended to be exhaustive or to limit the invention to the precise form disclosed, and modifications and variations are possible in light of the above teachings or may be acquired from practice of the invention. The drawings and description were chosen to explain the principles of the invention and its practical application. It is intended that the scope of the invention be defined by the claims appended hereto, and their equivalents.

1. A method of destroying documents comprising:
supplying the documents to an apparatus adapted to shred documents and medical waste, the apparatus having little or no liquid effluent and little or no toxic air emissions;
shredding the documents; and
discharging the shredded documents from the apparatus.

2. The method of claim 1, wherein the documents include protected health information.

3. The method of claim 2, wherein protected health information comprises demographic data that identifies an individual or for which there is a reasonable basis to believe said data can be used to identify the individual.

4. The method of claim 3, wherein the demographic data comprises:
the individual's physical health, mental health, or medical condition; or
health care services requested by, sought by, recommended to, administered to, and/or prescribed to the individual; or
the billing records for the provision of health care to the individual.

5. The method of claim 3, wherein the demographic data comprises:
names, geographic subdivisions smaller than a state, elements of dates for dates directly related to the individual, telephone numbers, fax numbers, electronic mail addresses, social security numbers, medical record numbers, health plan beneficiary numbers, account numbers, certificate/license numbers, vehicle identifiers and serial numbers, license plate numbers, device identifiers and serial numbers, web universal resource locators, internet protocol address numbers, biometric identifiers, or full face photographic images.

6. The method of claim 5, wherein geographic subdivisions smaller than a state includes street address, city, county, precinct, zip code, or their equivalent geocodes.

7. The method of claim 5, wherein elements of dates includes birth date, admission date, discharge date, date of death and age.

8. The method of claim 5, wherein biometric identifiers includes finger or voice prints.

9. The method of claim 1, further comprising supplying medical waste to the apparatus, prior to, concurrent with, or after supplying the documents.

10. The method of claim 9, further comprising mixing the documents with the medical waste in the apparatus.

11. The method of claim 10, wherein the medical waste comprises, pathological waste, sharps, bandages, rags, body fluids, cultures, stocks of etiological agents, IV bags, medicine bottles, diseased animal carcasses, diseased human bodies, or natural disaster victims.

12. The method of claim 1, further comprising mixing the documents with contaminated food in the apparatus.

13. The method of claim 1, further comprising mixing the documents with illegal drugs or expired pharmaceuticals.

14. The method of claim 1, further comprising the step of spraying the documents with water to moisten them.

15. The method of claim 14, further comprising heating the moistened documents with microwaves.

16. The method of claim 15, further comprising heating the moistened documents with conductive heat, or infrared heat.

17. The method of claim 15, further comprising preheating the documents prior to shredding.

18. The method of claim 15, further comprising subjecting the documents to X-rays or ultra violet light.

19. The method of claim 15, further comprising exposing the documents to ozone.

20. The method of claim 15, further comprising maintaining the documents in a temperature maintenance chamber at a sufficient temperature for a sufficient amount of time to kill or destroy little or all germs, bacteria, and viruses thereon.

21. The method of claim 1, further comprising compacting the shredded documents after the step of discharging the shredded documents from the shredding apparatus.

22. The method of claim 1, wherein the discharge from the shredding apparatus is suitable for landfill or recycling without additional treatment.

23. An apparatus for processing of documents and medical waste comprising;
a shredding mechanism;
a microwave chamber;
a temperature maintenance chamber; and
an ozone electrode,
wherein the apparatus has little or no liquid effluent and little or no toxic air emissions.

24. The apparatus of claim 23, further comprising a secondary radiation source in the temperature maintenance chamber.

25. The apparatus of claim 24, wherein the secondary radiation source emits X-rays or UV light.

26. A method of tracking medical or toxic waste comprising:
monitoring the movement of a first container having a wireless tracking device attached thereto from a waste generating facility to a waste treatment facility using the wireless tracking device.

27. The method of claim 1, further comprising:
monitoring the return of the first container from the waste treatment facility to the waste generating facility with the wireless tracking device.

28. The method of claim 2, wherein monitoring comprises scanning the wireless tracking device with a handheld reader.

29. The method of claim 3, further comprising uploading tracking data to a monitoring station.

30. The method of claim 1, wherein the medical waste is segregated according to type prior to putting it in the medical waste container.

31. The method of claim 5, further comprising weighing the first container with waste prior to treating the waste.

32. The method of claim 6, further comprising determining the amount of waste in the container.

33. The method of claim 7, further comprising calculating how much to charge a customer based on the type of waste and the weight of the waste.

34. The method of claim 8, further comprising electronically billing the customer.

35. The method of claim 9, further comprising supplying the customer with an invoice, the invoice including confirmation of the destruction of the waste and the amount of waste destroyed.

36. The method of claim 10, further comprising supplying a second container having an wireless tracking device, the second container adapted to hold treated medical waste.

37. The method of claim 11, further comprising tracking the second container to a landfill or recycling center.

38. The method of claim 12, further comprising supplying the customer with an invoice, the invoice including confirmation of the delivery of the waste to the landfill or recycling center.

39. The method of claim 13, wherein the wireless tracking device operates at radio frequencies.

40. A system for tracking medical or toxic waste comprising:
a first container adapted to hold untreated medical waste, the container having a wireless tracking device attached thereto; and
a tracking station capable of monitoring the movement of the medical waste by tracking the wireless tracking device.

41. The system of claim 15, further comprising at least one handheld reader.

42. The system of claim 15, further comprising a satellite or cellular station.

43. The system of claim 17, further comprising a computer to track said container.

44. The system of claim 18, further comprising an apparatus for weighing the first container and untreated medical waste.

45. The system of claim 19, further comprising an apparatus for calculating how much to charge a customer based on the type of waste and the weight of the waste and for supplying the customer with an invoice, the invoice including confirmation of the destruction of the waste and the amount of waste destroyed.

46. The system of claim 20, further comprising a second container having an wireless tracking device, the second container adapted to hold treated medical waste.

47. The system of claim 21, wherein the wireless tracking operates at radio frequencies.

48. (canceled)
